(19) United States
(12) Patent Application Publication
(10) Pub. No.: US 2024/0052318 A1
(43) Pub. Date: Feb. 15, 2024

(54) Methods for Differentiating AT2 Cells

(71) Applicant: Lung Biotechnology PBC, Silver Spring, MD (US)

(72) Inventors: Adrian COOPER, Silver Spring, MD (US); Andre DHARMAWAN, Silver Spring, MD (US); Elena FEDERZONI, Silver Spring, MD (US); Kurt WONG, Silver Spring, MD (US)

(21) Appl. No.: 18/354,201

(22) Filed: Jul. 18, 2023

Related U.S. Application Data
(60) Provisional application No. 63/390,463, filed on Jul. 19, 2022.

(51) Int. Cl. C12N 5/071

(52) U.S. Cl. CPC C12N 5/0688; C12N 2501/117; C12N 2501/119; C12N 2501/39; C12N 2501/999; C12N 2501/01

(57) ABSTRACT

The present disclosure relates to methods of differentiating pluripotent stem cells or lung progenitor cells into alveolar type 2 (AT2) cells. The present disclosure also relates to AT2 cells made by such methods, organoids containing such AT2 cells and methods of using the same. The present disclosure also relates to differentiation media for use in the same.

### CROSS REFERENCE TO RELATED APPLICATIONS

This application claims the priority benefit of U.S. Provisional Application No. 63/390,463, filed Jul. 19, 2022, which is incorporated herein by reference in its entirety.

### FIELD OF THE DISCLOSURE

The present disclosure relates to methods of differentiating pluripotent stem cells or lung progenitor cells into alveolar type 2 (AT2) cells. The present disclosure also relates to AT2 cells made by such methods, organoids containing such AT2 cells, and methods of using the same. The present disclosure also relates to differentiation media for use in the same.

### BACKGROUND

Pluripotent stem cells (PSC) are undifferentiated or partially differentiated cells that can differentiate into various other cell types. Induced pluripotent stem cells (iPSC) are a type of PSC derived from adult somatic cells that have been genetically reprogrammed to an embryonic stem cell (ESC)-like state through the expression of genes and factors important for maintaining the defining properties of ESC. iPSC have generated interest in the medical community recently because they address many obstacles associated with the use of embryonic stem cells, and allow for the generation of patient-specific PSC, which can be genetically corrected, differentiated into adult lineages, and returned to the same patient as an autograft. Yamanaka et al., Cell Stem Cell. 1(1):39-49 (2007); Nishikawa et al., Nat. Rev. Mol. Cell Biol. 9:725 (2008). In addition to genetic disorders, iPSC can be used for tissue regeneration and disease modeling. Kogut et al., Methods Mol. Biol. 1195:1-12 (2014).

### BRIEF SUMMARY

The present disclosure provides methods for differentiating pluripotent stem cells (PSC) or lung progenitor cells (LPC) into alveolar type 2 (AT2) cells.

In some aspects, the method comprises (i) culturing LPC in a base culture medium comprising a glycogen synthase kinase 3 (GSK3) inhibitor, keratinocyte growth factor (KGF), fibroblast growth factor 10 (FGF10), and a gamma secretase inhibitor; (ii) passaging the cells of (i) in a base culture medium comprising a GSK3 inhibitor, KGF, FGF10, dexamethasone, cyclic adenosine monophosphate (cAMP), an inhibitor of cyclic nucleotide phosphodiesterases, and a Rho-associated, coiled-coil containing protein kinase (ROCK) inhibitor for about 24 hours to about 48 hours; (iii) culturing the cells of (ii) in a base culture medium comprising a GSK3 inhibitor, KGF, FGF10, dexamethasone, cyclic adenosine monophosphate (cAMP), and an inhibitor of cyclic nucleotide phosphodiesterases for about 7 days; (iv) separating the cells of (iii) having expression of epithelial cellular adhesion molecule (EpCAM) and/or carboxypeptidase M (CPM); (v) passaging the cells of (iv) having expression of EpCAM and/or CPM in a base culture medium comprising a GSK3 inhibitor, KGF, FGF10, dexamethasone, cAMP, and an inhibitor of cyclic nucleotide phosphodiesterases, and a ROCK inhibitor; and (vi) separating the cells of (v) having expression of surfactant protein C (SFTPC) to form AT2 cells.

In some aspects, the passaging of (ii) is performed in a two dimensional (2D) matrix. In some aspects, the 2D matrix is Matrigel®.

In some aspects, the passaging of (v) is performed in a three dimensional (3D) matrix. In some aspects, the 3D matrix is Matrigel®.

In some aspects, the method comprises (i) culturing LPC in a base culture medium comprising a GSK3 inhibitor, KGF, FGF10, and a gamma secretase inhibitor; (ii) separating the cells of (i) having expression of EpCAM and/or CPM; (iii) passaging the cells of (ii) having expression of EpCAM and/or CPM in a base culture medium comprising a GSK3 inhibitor, KGF, FGF10, dexamethasone, cAMP, an inhibitor of cyclic nucleotide phosphodiesterases, and a ROCK inhibitor for about 24 hours to about 48 hours; (iv) culturing the cells of (iii) in a base culture medium comprising a GSK3 inhibitor, KGF, FGF10, dexamethasone, cAMP, and an inhibitor of cyclic nucleotide phosphodiesterases for about 7 days, wherein the base culture medium does not comprise or is essentially free of a ROCK inhibitor; and (v) separating the cells of (iv) having expression of SFTPC to form AT2 cells.

In some aspects, the passaging of (iii) is performed in a 3D matrix. In some aspects, the 3D matrix is Matrigel®.

In some aspects, the method comprises (i) culturing LPC in a base culture medium comprising a GSK3 inhibitor, KGF, FGF10, and a gamma secretase inhibitor; (ii) passaging the cells of (i) in a base culture medium comprising a GSK3 inhibitor, KGF, FGF10, dexamethasone, cAMP, an inhibitor of cyclic nucleotide phosphodiesterases, and a ROCK inhibitor; (iii) separating the cells of (ii) having expression of EpCAM and/or CPM; (iv) passaging the cells of (iii) having expression of EpCAM and/or CPM in a base culture medium comprising KGF, FGF10, dexamethasone, cAMP, an inhibitor of cyclic nucleotide phosphodiesterases, and a ROCK inhibitor; (v) culturing the cells of (iv) in a base culture medium comprising KGF, FGF10, dexamethasone, cAMP, an inhibitor of cyclic nucleotide phosphodiesterases, and a GSK3 inhibitor, wherein the base culture medium does not comprise or is essentially free of a ROCK inhibitor; and (vi) separating the cells of (v) having expression of SFTPC to form AT2 cells.

In some aspects, the passaging of (iv) is from about 2 days to about 4 days and/or the culturing of (v) is from about 2 days to about 4 days.

In some aspects, the passaging of (iv) is for about 2 days and/or the culturing of (v) is for about 2 days.

In some aspects, the passaging of (iv) and/or the culturing of (v) are repeated before the separating of (vi).

In some aspects, the repeated passaging of (iv) is for about 4 days and the repeated culturing of (v) is for about 4 days.

In some aspects, the passaging of (ii) is performed in a 2D matrix. In some aspects, the 2D matrix is Matrigel®.

In some aspects, the passaging of (iv) is performed in a 3D matrix. In some aspects, the 3D matrix is Matrigel®.

In some aspects, the GSK3 inhibitor is CHIR99021. In some aspects, CHIR99021 is present in the culture medium at a concentration of about 3 μM.

In some aspects, FGF10 is present in the culture medium at a concentration of about 10 ng/mL.

In some aspects, KGF is present in the culture medium at a concentration of about 10 ng/mL.

In some aspects, the gamma secretase inhibitor is N-[N-(3,5-difluorophenacetyl)-1-alanyl]-s-phenylglycinet-butyl ester (DAPT). In some aspects, DAPT is present in the culture medium at a concentration of about 20 μM.

In some aspects, dexamethasone is present in the culture medium at a concentration of about 50 nM.

In some aspects, cAMP is present in the culture medium at a concentration of about 100 μM.

In some aspects, the inhibitor of cyclic nucleotide phosphodiesterases is 3-isobutyl-1-methylxanthine (IBMX). In some aspects, IBMX is present in the culture medium at a concentration of about 100 μM.

In some aspects, the ROCK inhibitor is Y-27632. In some aspects, Y-27632 is present in the culture medium at a concentration of about 10 μM.

The present disclosure also provides AT2 cells made by a differentiation method disclosed herein, an organoid comprising AT2 cells disclosed herein, and certain methods of use thereof.

The present disclosure also provides certain differentiation media. In some aspects, the differentiation medium comprises a base culture medium, a GSK3 inhibitor, KGF, FGF10, dexamethasone, cAMP, an inhibitor of cyclic nucleotide phosphodiesterases, and a ROCK inhibitor. In some aspects, the differentiation medium comprises a base culture medium, about 3 μM CHIR99021, about 10 ng/mL KGF, about 10 ng/mL FGF10, about 50 nM dexamethasone, about 100 μM cAMP, about 100 μM IBMX, and about 10 μM Y-27632.

The present disclosure also provides a differentiation medium comprising a base culture medium, KGF, FGF10, dexamethasone, cAMP, an inhibitor of cyclic nucleotide phosphodiesterases, and a ROCK inhibitor. In some aspects, the differentiation medium comprises a base culture medium, about 10 ng/mL KGF, about 10 ng/mL FGF10, about 50 nM dexamethasone, about 100 μM cAMP, about 100 μM IBMX, and about 10 μM Y-27632.

### BRIEF DESCRIPTION OF THE DRAWINGS

Some aspects of the invention are herein described, by way of example only, with reference to the accompanying drawings. With specific reference now to the drawings in detail, it is stressed that the particulars shown are by way of example and for purposes of illustrative discussion of aspects of the invention.

FIG. 1 shows flow cytometry results for SFTPC reporter expression of cells from day 17, day 34, day 45, day 59, day 72, and day 84 of AT2 differentiation.

FIG. 2A-2B shows expression of AT2-associated genes during differentiation relative to a primary AT2 cell control. FIG. 2A shows expression of SFTPC. FIG. 2B shows expression of LPCAT1.

FIG. 3 shows expression of SFTPC protein at day 94 of differentiated cells (iPS-AT2) and control primary AT2 cells.

### DETAILED DESCRIPTION

### I. General Definitions

Unless defined otherwise, all technical and scientific terms used herein have the same meaning as commonly understood by one of ordinary skill in the art to which this disclosure belongs. In case of conflict, the present application, including the definitions, will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range. Numeric ranges recited are inclusive of the numbers defining the range and include each integer within the defined range.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

### II. Differentiation Methods

The present disclosure relates to improved methods of differentiating pluripotent stem cells (PSC) or lung progenitor cells (LPC) into alveolar type 2 (AT2) cells. Such methods result, for example, in improved cell viability, yield and/or characteristics of the differentiated cells.

As used herein, the terms “differentiation” and “differentiating” refer to the process of inducing or reprogramming young or immature cells (e.g., pluripotent stem cells) into more mature or specialized cells (e.g., AT2 cells). In general, differentiation of pluripotent stem cells can be effected, for example, by changing culturing conditions of the cells, such as changing the stimuli agents in a culture medium or the physical state of the cells.

As used herein, the terms “pluripotent stem cell,” “pluripotent stem cells,” and “PSC” refer to young or immature cell(s) that can develop into more mature or specialized cells (e.g., AT2 cells).

In some aspects, PSC include, but are not limited to, embryonic stem cells (ESC), induced pluripotent stem cells (iPSC), embryonic germ cells, adult stem cells, or a combination thereof. In some aspects, the PSC are from a human. In some aspects, the PSC are from an animal. In some aspects, the animal is a sheep, pig or primate.

As used herein, the terms “induced pluripotent stem cell,” “induced pluripotent stem cells,” and “iPSC” refer to cells produced from differentiated adult, neonatal or fetal cells that have been induced or reprogrammed into pluripotent stem cells.

As used herein, the terms “lung progenitor cell,” “lung progenitor cells,” or “LPC” refer to pluripotent cells capable of differentiating into several cell types of the respiratory system, including, but not limited to, pneumocyte type 1 and type II cells, interalveolar cells, smooth muscle cells, alveoli epithelial cells, endothelial cells and erythrocytes. LPC are committed to the pulmonary lineage, retain the ability to self-renew, and can be identified, for example, by cell surface markers or intracellular proteins that include, but are not limited to, transcription termination factor 1 (TTF1), GATA binding protein 6 (GATA6), Est1, Nkx2.1, surfactant protein C (SP-C), forkhead box A1 (FOXA1), FOXA2, SRY-box transcription factor 2 (SOX2), SOX9, CD49f, cytokeratin 8 (CK8), epithelial cellular adhesion molecule (EpCAM), and p63. In some aspects, LPC according to the present disclosure do not express certain markers or exhibit negative or low expression of certain markers, such as markers of endothelial cells (e.g., CD144, CD31), markers of hematopoietic cells (e.g., CD43, CD45, CD235a, or CD41a) and/or markers of pluripotent stem cells (e.g., TRA1-60).

In some aspects, the LPC are derived from PSC (e.g., iPSC). Methods for generating LPC from PSC (e.g., iPSC) are known and described further herein and, for example, in Jacob et al., Nature Protocols, 14:3303-3332, 2019; Hawkins et al., Cell Stem Cell, 28:79-95, 2021.

As used herein, the terms “alveolar type II cells,” “alveolar type II cell,” “AT2 cells,” or “AT2 cell” refer to cells that line lung alveoli and produce surfactants which allow for appropriate gas exchange through lung inflation (e.g., surfactant protein C (SFTPC)). AT2 cells contain electron-dense organelles with a lamellar structure (lamellar bodies). As used herein, an AT2 cell includes a mature AT2 cell, an AT2 progenitor cell, and an AT2 precursor cell.

In some aspects, AT2 cells made by the differentiation methods provided herein express one or more markers (e.g., cell surface markers, mRNAs, proteins, epigenetic signatures) typical of AT2 cells. Examples of such markers include, but are not limited to, Nkx2.1, SFTPC, SFTPB, lysophosphatidylcholine acyltransferase 1 (LPCAT1), SFTPA2, SFTPD, ATP Binding Cassette Subfamily A Member 2 (ABCA2), napsin A aspartic peptidase 1 (NAPSA1), progastricsin (PGC), Solute Carrier Family 34 Member 2 (SLC34A2), epithelial cellular adhesion molecule (EpCAM), and Carboxypeptidase M (CPM). In some aspects, the marker is EpCAM. In some aspects, the marker is CPM. In some aspects, the marker is SFTPC.

As used herein, the terms “expression,” “expresses,” or “express” with regard to a marker (e.g., EpCAM, CPM and/or SFTPC) include, but are not limited to, detectable expression of the marker, expression of the marker comparable to or greater than mature AT2 cells, or precursors or progenitors thereof, and expression of the marker that is greater than a control cell that does not express the marker and/or is not an AT2 cell.

In some aspects, AT2 cells made by the differentiation methods provided herein have certain morphological features typical of AT2 cells. Examples of such features include, but are not limited to, a cuboidal shape, lamellar bodies and/or microvilli, when analyzed, for example, with a microscope, e.g., an electron microscope.

In some aspects, the differentiation methods provided herein include certain cell culturing conditions, such as culturing cells in certain culture media.

As used herein, the terms “cell culture,” “cell culturing,” “culture,” “culturing,” and “cultured” refer to the maintenance, growth and/or differentiation of cells in an in vitro environment. The terms “cell culture medium,” “cell culture media,” “culture medium” and “culture media” refer to a composition for culturing cells that contains nutrients to maintain cell viability, support proliferation and optionally differentiation. A cell culture medium can contain one or more of the following: salt(s), buffer(s), amino acid(s), glucose or other sugar(s), antibiotic(s), serum or serum replacement, and other components such as growth factors, vitamins, etc.

In some aspects, the differentiation methods provided herein refer to a cell culture media as a “base culture medium” supplemented with other components. As used herein, a “base culture medium” or “base culture media” refer to a composition that contains the minimal elements required for maintenance, growth and/or differentiation of cells in an in vitro environment. Examples of a base culture medium include, but are not limited to, Dulbecco's Modified Eagle Medium (DMEM), MEM, Iscove's Modified Dulbecco's Medium (IMDM), Glasgow's modified MEM (GMEM), DMEM/F12, Leibovitz L-15, RPMI-1640, CMRL, Ham F10, and Ham F12. In some aspects, a base culture medium is supplemented with one or more other components such as amino acid(s), antibiotic(s), serum, growth factor(s), etc. Such components are well known in the art and described further herein.

In some aspects, a cell culture medium or base culture medium of the differentiation methods provided is “essentially free” of or “does not comprise” a certain component (e.g., a ROCK inhibitor such as Y-27632). As used herein, the term “essentially free” refers to a culture medium that is at least 95% free, 96% free, 97% free, 98% free, or 99% free of a certain component; or has an undetectable amount of the component (e.g., a ROCK inhibitor such as Y-27632), as measured by methods known in the art and described further herein. The terms “does not comprise” and “do not comprise” refer to a culture medium that does not contain a certain component (e.g., a ROCK inhibitor such as Y-27632), or has an undetectable amount of the component, as measured by methods known in the art and described further herein.

In some aspects, the differentiation methods provided herein include certain cell culturing conditions, such passaging cells in certain culture medium. As used herein, the terms “passage,” “passaged,” and “passaging” refer to the act of subdividing and plating cells at a lower concentration into one or more cell culture surfaces or vessels, when the cells have proliferated to a desired extent. Passaging typically involves detaching cells by mechanical or enzymatic methods (e.g., incubation in Accutane®) before plating, optionally at a certain cell density. Methods for passaging cells are well known and described further herein.

In some aspects, culturing and passaging in the differentiation methods provided herein are performed with one or more substrates coated onto the cell culture surface or vessel. Such substrates include, but are not limited to, vitronectin, gelatin, laminin (e.g., laminin-111, laminin-211, laminin-121, laminin-221, laminin-332, laminin-311, laminin-321, laminin-411, laminin-421, laminin-511, laminin-213, laminin-521, laminin-423, laminin-522, laminin-523, or a combination thereof), fibronectin, collagen (e.g., collagen I, collagen IV, or a combination thereof), elastin, osteopontin, thrombospondin, mixtures of naturally occurring cell line-produced matrices such as Matrigel™, and synthetic or man-made surfaces such as polyamine monolayers and carboxy-terminated monolayers, or a combination thereof. In some aspects, the substrate is laminin-521. Methods for coating a substrate onto a cell culture surface or vessel are well known and described further herein.

In some aspects, culturing and/or passaging of the differentiation methods provided herein are in a two dimensional (2D) matrix. In such aspects, cells are grown as a monolayer on a cell culture surface or vessel, optionally with a substrate coating described above.

In some aspects, culturing and/or passaging of the differentiation methods provided herein are in a three dimensional (3D) matrix. Examples of a 3D matrix include, but are not limited to, polymers (natural or synthetic), ceramics, composites, and a combination thereof. A 3D matrix can be in the form of a hydrogel, a porous 3D scaffold, a rapid-prototyping scaffold, a foam, a sponge, a mesh, microparticles, fiber-like networks, mixtures of naturally occurring cell line-produced matrices such as Matrigel™ and a combination thereof, for example, microparticle-loaded hydrogels.

In some aspects, the differentiation methods provided herein include one or more steps where cultured cells having certain biochemical characteristics are separated. As used herein, the terms “separated” and “separating” refer to a process of isolating one or more specific cell populations from a heterogeneous mixture of cells.

In some aspects, the differentiation methods provided herein include separating cells having expression of EpCAM and/or CPM. In some aspects, the differentiation methods provided herein include separating cells having expression of SFTPC.

Cell separation methods based on expression of a marker are well known in the art and include, but are not limited to, affinity separation, fluorescence-activated cell sorting (FACS), density gradient centrifugation, immunodensity cell isolation, microfluidic cell sorting, buoyancy-activated cell sorting, aptamer-based cell isolation, complement depletion, and more. Techniques for affinity separation include, but are not limited to, separation using antibody-coated magnetic beads (e.g., immunomagnetic cell separation), affinity chromatography, cytotoxic agents joined to a monoclonal antibody or used in conjunction with a monoclonal antibody, e.g., complement and cytotoxins, and “panning” with an antibody attached to a solid matrix, e.g. plate, or other convenient technique. In some aspects, cells of the present differentiation methods are separated by immunomagnetic cell separation.

Some aspects of the differentiation methods provided herein include culturing and/or passaging cells in a base culture medium containing a glycogen synthase kinase 3 (GSK3) inhibitor. GSK3 is a serine/threonine protein kinase that mediates the addition of phosphate molecules on certain serine and threonine amino acids of cellular substrates (e.g., glycogen synthase). This phosphorylation typically results in inhibition of the substrates. GSK3 has also been implicated in the control of cellular response to damaged DNA and Wnt signaling and phosphorylation of Ci in the Hedgehog (Hh) pathway, targeting it for proteolysis to an inactive form.

As used herein, a “GSK3 inhibitor” refers to a compound that inhibits one or more GSK3 enzymes. The family of GSK3 enzymes is well known and a number of variants have been described (e.g., Schaffer et al., Gene, 302:73-81, 2003). Specific examples of GSK3 inhibitors include, but are not limited to, Kenpaullone, 1-Azakenpaullone, CHIR99021, CHIR98014, AR-A014418, CT99021, CT20026, SB415286, SB216763, AR-A014418, lithium, SB 415286, and TDZD-8. Further exemplary GSK3 inhibitors include, but are not limited to, BIO (2′Z,3′£)-6-Bromomdirubm-3′-oxime (GSK3 Inhibitor IX); BIO-Acetoxime (2′Z,3′E)-6-Bromoindirubin-3′-acetoxime (GSK3 Inhibitor X); (5-Methyl-1H-pyrazol-3-yl)-(2-phenylquinazolin-4-yl)amine (GSK3 Inhibitor XIII); Pyridocarbazole-cyclopenadienylruthenium complex (GSK3 Inhibitor XV); TDZD-8,4-Benzyl-2-methyl-1,2,4-thiadiazolidine-3,5-dione (GSK3beta Inhibitor I); 2-Thio(3-iodobenzyl)-5-(1-pyridyl)-[1,3,4]-oxadiazole (GSK3beta Inhibitor II); OTDZT 2,4-Dibenzyl-5-oxothiadiazolidine-3-thione (GSK3beta Inhibitor III); alpha-4-Dibromoacetophenone (GSK3beta Inhibitor VII); AR-AO 14418 N-(4-Methoxybenzyl)-N′-(5-nitro-1,3-thiazol-2-yl)urea (GSK-3beta Inhibitor VIII); 3-(1-(3-Hydroxypropyl)-1H-pyrrolo[2,3-b]pyridin-3-yl]-4-pyrazin-2-yl-pyrrole-2,5-dione (GSK3beta Inhibitor XI); TWS1 19-pyrrolopyrimidine compound (GSK3beta Inhibitor XII); L803 H-KEAPP APPQSpP-NH2 or its Myristoylated form (GSK3beta Inhibitor XIII); 2-Chloro-1-(4,5-dibromo-thiophen-2-yl)-ethanone (GSK3beta Inhibitor VI); AR-AO 144-18; SB216763; and SB415286. In some aspects, the GSK3 inhibitor is CHIR99021.

In some aspects, the GSK3 inhibitor (e.g., CHIR99021) is present in the base culture medium at a concentration of from about 0.5 μM to about 61.1M, or any value or range of values thereof, including, for example, from about 11.1M to about 6 μM, from about 3 μM to about 6 μM, from about 0.5 μM to about 3 μM, from about 11.1M to about 3 μM, or from about 0.5 μM to about 1 μM. In some aspects, the GSK3 inhibitor (e.g., CHIR99021) is about 0.5 μM, about 11.1M, about 3 μM or about 6 μM. In some aspects, the GSK3 inhibitor (e.g., CHIR99021) is about 3 μM.

Some aspects of the differentiation methods provided herein include culturing and/or passaging cells in a base culture medium containing keratinocyte growth factor (KGF). KGF is a member of the heparin-binding fibroblast growth factor family with a distinctive pattern of target-cell specificity. KGF is mitogenically active on epithelial cells and produced by cells of mesenchymal origin.

In some aspects, KGF is present in the base culture medium at a concentration of from about 2 ng/mL to about 20 ng/mL, or any value or range of values thereof, including, for example, from about 5 ng/mL to about 20 ng/mL, from about 10 ng/mL to about 20 ng/mL, from about 2 ng/mL to about 10 ng/mL, from about 5 ng/mL to about 10 ng/mL, or from about 2 ng/mL to about 5 ng/mL. In some aspects, KGF is present in the base culture medium at a concentration of about 2 ng/mL, about 5 ng/mL, about 10 ng/mL, or about 20 ng/mL. In some aspects, KGF is present in the base culture medium at a concentration of about 10 ng/mL.

Some aspects of the differentiation methods provided herein include culturing and/or passaging cells in a base culture medium containing fibroblast growth factor 10 (FGF10). FGF10 is a paracrine signaling molecule seen first in the limb bud and organogenesis development. FGF10 starts the developing of limbs and is involved in the branching of morphogenesis in multiple organs such as the lungs, skin, ear and salivary glands.

In some aspects, FGF10 is present in the base culture medium at a concentration of from about 2 ng/mL to about 20 ng/mL, or any value or range of values thereof, including, for example, from about 5 ng/mL to about 20 ng/mL, from about 10 ng/mL to about 20 ng/mL, from about 2 ng/mL to about 10 ng/mL, from about 5 ng/mL to about 10 ng/mL, or from about 2 ng/mL to about 5 ng/mL. In some aspects, FGF10 is present in the base culture medium at a concentration of about 2 ng/mL, about 5 ng/mL, about 10 ng/mL, or about 20 ng/mL. In some aspects, FGF10 is present in the base culture medium at a concentration of about 10 ng/mL.

Some aspects of the differentiation methods provided herein include culturing and/or passaging cells in a base culture medium containing a gamma secretase inhibitor. As used herein, the term “gamma secretase” refers to any protein or protein complex that exhibits gamma secretase activities, including, but not limited to, binding to a substrate having a gamma secretase cleavage sequence, and catalyzing the cleavage of the gamma secretase cleavage sequence, at a gamma secretase cleavage site, to produce substrate cleavage products. In some aspects, gamma secretase is a protein complex comprising one or more of the following subunits: presenilin, nicastrin, gamma secretase subunit APH-1, and gamma secretase subunit PEN-2.

As used herein, the terms “gamma secretase inhibitor,” “gamma secretase inhibitors” or “GSI” refer to any material or compound that, e.g., binds to, partially or totally blocks activity, decreases, prevents, delays activation, inactivates, desensitizes, or downregulates the activity or expression of gamma secretase or the gamma secretase pathway. Examples of a gamma secretase inhibitor include, but are not limited to, genetically modified versions of gamma secretase proteins, e.g., versions with altered activity, as well as naturally occurring and synthetic ligands, antagonists, agonists, antibodies, small chemical molecules and the like. In some aspects, the gamma secretase inhibitor reduces expression and/or function of a subunit of gamma secretase (e.g., presenilin, nicastrin, APH-1, or PEN-2).

More specific examples of gamma secretase inhibitors include, but are not limited to, DAPT (N-[N-(3,5-difluorophenacetyl-L-alanyl)]-S-phenylglycine t-butyl ester), talsaclidine (Hock et al., 2003), Xanomeline, L-689660, L-685458, McN-A-343, CDD-0097, fenchylamine, MG132, WPE-111-31C, MW-11-36C/26A, MW-167, CM-265, lactacystin, DNPS1, DAM, LY-450139, PF-5212362, BMS-708163, MK-0752, ELN-318463, BMS-299897, LY-411575, BMS-906024, PF-3084014, R04929097, and LY3039478. In some aspects, the gamma secretase inhibitor is DAPT.

Some aspects of the differentiation methods provided herein include culturing and/or passaging cells in a base culture medium containing dexamethasone. Dexamethasone is an anti-inflammatory glucocorticoid with a range of effects on cell proliferation and differentiation.

In some aspects, dexamethasone is present in the base culture medium at a concentration of from about 20 nM to about 80 nM, or any value or range of values thereof, including, for example, from about 40 nM to about 80 nM, from about 50 nM to about 80 nM, from about 60 nM to about 80 nM, from about 20 nM to about 60 nM, from about 40 nM to about 60 nM, from about 50 nM to about 60 nM, from about 20 nM to about 50 nM, from about 40 nM to about 50 nM, or about 20 nM to about 40 nM. In some aspects, dexamethasone is present in the base culture medium at a concentration of about 20 nM, about 40 nM, about 50 nM, about 60 nM, or about 80 nM. In some aspects, dexamethasone is present in the base culture medium at a concentration of about 50 nM.

Some aspects of the differentiation methods provided herein include culturing and/or passaging cells in a base culture medium comprising cyclic adenosine monophosphate (cAMP). cAMP facilitates mobilization of glucose and fatty acid reserves and is involved in the function and differentiation of many different cells types.

In some aspects, cAMP is present in the base culture medium at a concentration of from about 25 μM to about 200 μM, or any value or range of values thereof, including, for example, from about 50 μM to about 200 μM, from about 100 μM to about 200 μM, from about 150 μM to about 200 μM, from about 25 μM to about 150 μM, from about 50 μM to about 150 μM, from about 100 μM to about 150 μM, from about 25 μM to about 100 μM, from about 50 μM to about 100 μM, or from about 25 μM to about 50 μM. In some aspects, cAMP is present in the base culture medium at a concentration of about 25 μM, 50 μM, 100 μM, 150 μM, or 200 μM. In some aspects, cAMP is present in the base culture medium at a concentration of about 100 μM.

Some aspects of the differentiation methods provided herein include culturing and/or passaging cells in a base culture medium comprising an inhibitor of cyclic nucleotide phosphodiesterases. Cyclic nucleotide phosphodiesterases are a family of enzymes that hydrolyze the phosphodiester bond in cyclic adenosine monophosphate and cyclic guanosine monophosphate, thereby inhibiting their pulmonary vasodilator properties. In some aspects, the inhibitor of cyclic nucleotide phosphodiesterases is theophylline, 3-isobutyl-1-methylxanthine (IBMX), or Ro 20-1724. In some aspects, the inhibitor of cyclic nucleotide phosphodiesterases is IBMX.

In some aspects, the inhibitor of cyclic nucleotide phosphodiesterases (e.g., IBMX) is present in the base culture medium at a concentration of from about 25 μM to about 200 μM, or any value or range of values thereof, including, for example, from about 50 μM to about 200 μM, from about 100 μM to about 200 μM, from about 150 μM to about 200 μM, from about 25 μM to about 150 μM, from about 50 μM to about 150 μM, from about 100 μM to about 150 μM, from about 25 μM to about 100 μM, from about 50 μM to about 100 μM, or from about 25 μM to about 50 μM. In some aspects, the inhibitor of cyclic nucleotide phosphodiesterases (e.g., IBMX) is present in the base culture medium at a concentration of about 25 μM, 50 μM, 100 μM, 150 μM, or 200 μM. In some aspects, the inhibitor of cyclic nucleotide phosphodiesterases (e.g., IBMX) is present in the base culture medium at a concentration of about 100 μM.

Some aspects of the differentiation methods provided herein include culturing and/or passaging cells in a base culture medium containing a Rho associated kinase (ROCK) inhibitor. ROCK is a serine/threonine kinase that serves downstream effectors of Rho kinases, of which three isoforms exist (RhoA, RhoB and RhoC). A “ROCK inhibitor” can, for example, decrease ROCK expression and/or ROCK activity. Examples of a ROCK inhibitor include, but are not limited to, polynucleotides, polypeptides, and small molecules. More specific examples of a ROCK inhibitor include, but are not limited to, an anti-ROCK antibody, and dominant-negative ROCK variant, siRNA, shRNA, miRNA and antisense nucleic acids that target ROCK. Other examples of a ROCK inhibitor include, but are not limited to, thiazovivin, Y-27632, Fasudil, AR122-86, Y-30141, WF-536, HA-1077, hydroxyl-HA-1077, GSK269962A, SB-772077-B, N-(4-Pyridyl)-N′-(2,4,6-trichlorophenyl)urea, 3-(4-Pyridyl)-1H-indole, (R)-(+)-trans-N-(4-Pyridyl)-4-(1-aminoethyl)-cyclohexanecarboxamide, and ROCK inhibitors disclosed in U.S. Pat. No. 8,044,201, which is herein incorporated by reference in its entirety. In some aspects, the ROCK inhibitor is Y-27632.

In some aspects, the ROCK inhibitor (e.g., Y-27632) is present in the base culture medium at a concentration of from about 1 μM to about 20 μM, or any value or range of values thereof, including, for example, from about 1 μM to about 15 μM, from about 1 μM to about 10 μM, from about 1 μM to about 5 μM, from about 5 μM to about 20 μM, from about 5 μM to about 15 μM, from about 5 μM to about 10 μM, from about 10 μM to about 20 μM, from about 10 μM to about 15 μM, or from about 15 μM to about 20 μM. In some aspects, the ROCK inhibitor (e.g., Y-27632) is present in the base culture medium at a concentration of about 1 μM, about 5 μM, about 10 μM, about 15 μM, or about 20 μM. In some aspects, the ROCK inhibitor (e.g., Y-27632) is present in the base culture medium at a concentration of about 10 μM.

In some aspects, the gamma secretase inhibitor (e.g., DAPT) is present in the base culture medium at a concentration of from about 5 μM to about 40 μM, or any value or range of values thereof, including, for example, from about 10 μM to about 40 μM, from about 20 μM to about 40 μM, from about 5 μM to about 20 μM, from about 10 μM to about 20 μM, or from about 5 μM to about 10 μM. In some aspects, the gamma secretase inhibitor (e.g., DAPT) is present in the culture medium at a concentration of about 5 μM, about 10 μM, about 20 μM or about 40 μM. In some aspects, the gamma secretase inhibitor (e.g., DAPT) is present in the base culture medium at a concentration of about 20 μM.

In some aspects, the differentiation methods provided herein include culturing and/or passaging cells in a base culture medium containing a GSK3 inhibitor (e.g., CHIR99021), KGF, FGF10, and a gamma secretase inhibitor (e.g., DAPT). In some aspects, the base culture medium contains from about 0.5 μM to about 61.1M of the GSK3 inhibitor (e.g., CHIR99021), from about 2 ng/mL to about 20 ng/mL KGF, from about 2 ng/mL to about 20 ng/mL FGF10, and from about 5 μM to about 40 μM of the gamma secretase inhibitor (e.g., DAPT). In some aspects, the base culture medium contains about 3 μM of the GSK3 inhibitor (e.g., CHIR99021), about 10 ng/mL KGF, about 10 ng/mL FGF10, and about 20 μM of the gamma secretase inhibitor (e.g., DAPT).

In some aspects, the differentiation methods provided herein include culturing and/or passaging cells in a base culture medium containing a GSK3 inhibitor (e.g., CHIR99021), KGF, FGF10, dexamethasone, cAMP, an inhibitor of cyclic nucleotide phosphodiesterases (e.g., IBMX), and a ROCK inhibitor (e.g., Y-27632). In some aspects, the base culture medium contains from about 0.5 μM to about 61.1M of the GSK3 inhibitor (e.g., CHIR99021), from about 2 ng/mL to about 20 ng/mL KGF, from about 2 ng/mL to about 20 ng/mL FGF10, from about 20 nM to about 80 nM dexamethasone, from about 25 μM to about 200 μM of cAMP, from about 25 μM to about 200 μM of the inhibitor of cyclic nucleotide phosphodiesterases (e.g., IBMX), and from about 1 μM to about 20 μM of the ROCK inhibitor (e.g., Y-27632). In some aspects, the base culture medium contains about 3 μM of the GSK3 inhibitor (e.g., CHIR99021), about 10 ng/mL KGF, about 10 ng/mL FGF10, about 50 nM dexamethasone, about 100 μM cAMP, about 100 μM of the inhibitor of cyclic nucleotide phosphodiesterases (e.g., IBMX), and about 10 μM of the ROCK inhibitor (e.g., Y-27632). In some aspects, the culturing and/or passaging in such a culture medium is for from about 24 hours to about 48 hours, or any values or range of values therein, such as, for example, from about 24 hours to about 36 hours, from about 24 hours to about 30 hours, from about 30 hours to about 48 hours, from about 30 hours to about 36 hours, or from about 36 hours to about 48 hours. In some aspects, the culturing and/or passaging is for about 24 hours, about 30 hours, about 36 hours, or about 48 hours.

In some aspects, the differentiation methods provided herein include culturing and/or passaging cells in a base culture medium containing a GSK3 inhibitor (e.g., CHIR99021), KGF, FGF10, dexamethasone, cAMP, and an inhibitor of cyclic nucleotide phosphodiesterases (e.g., IBMX). In some aspects, the base culture medium contains from about 0.5 μM to about 6 μM of the GSK3 inhibitor (e.g., CHIR99021), from about 2 ng/mL to about 20 ng/mL KGF, from about 2 ng/mL to about 20 ng/mL FGF10, from about 20 nM to about 80 nM dexamethasone, from about 25 μM to about 200 μM cAMP, and from about 25 μM to about 200 μM of the inhibitor of cyclic nucleotide phosphodiesterases (e.g., IBMX). In some aspects, the base culture medium contains about 3 μM of the GSK3 inhibitor (e.g., CHIR99021), about 10 ng/mL KGF, about 10 ng/mL FGF10, about 50 nM dexamethasone, about 100 μM cAMP, and about 100 μM of the inhibitor of cyclic nucleotide phosphodiesterases (e.g., IBMX). In some aspects, the culturing and/or passaging in such a culture medium is for from about 3 days to about 10 days, or any value or range of values therein, such as, for example, from about 5 days to about 10 days, from about 7 days to about 10 days, from about 5 days to about 10 days, from about 7 days to about 10 days, or from about 7 days to about 10 days. In some aspects, the culturing and/or passaging is for about 3 days, about 5 days, about 7 days, or about 10 days.

In some aspects, the differentiation methods provided herein comprise passaging cells in a base culture medium comprising a GSK3 inhibitor (e.g., CHIR99021), KGF, FGF10, dexamethasone, cAMP, an inhibitor of cyclic nucleotide phosphodiesterases (e.g., IBMX), and a ROCK inhibitor (e.g., Y-27632). In some aspects, the passaging is for about 24 hours to about 48 hours.

In some aspects, the differentiation methods provided herein comprise:
(i) culturing LPC in a base culture medium comprising a GSK3 inhibitor (e.g., CHIR99021), KGF, FGF10, and a gamma-secretase inhibitor (e.g., DAPT);
(ii) passaging the cells of (i) in a base culture medium comprising a GSK3 inhibitor (e.g., CHIR99021), KGF, FGF10, dexamethasone, cAMP, an inhibitor of cyclic nucleotide phosphodiesterases (e.g., IBMX), and a ROCK inhibitor (e.g., Y-27632);
(iii) culturing the cells of (ii) in a base culture medium comprising a GSK3 inhibitor, KGF, FGF10, dexamethasone, cAMP, and an inhibitor of cyclic nucleotide phosphodiesterases (e.g., IBMX);
(iv) separating the cells of (iii) having expression of EpCAM and/or CPM;
(v) passaging the cells of (iv) having expression of EpCAM and/or CPM in a base culture medium comprising a GSK3 inhibitor (e.g., CHIR99021), KGF, FGF10, dexamethasone, cAMP, and an inhibitor of cyclic nucleotide phosphodiesterases (e.g., IBMX), and a ROCK inhibitor (e.g., Y-27632); and
(vi) separating the cells of (v) having expression of SFTPC to form AT2 cells.

In some aspects, the passaging of (ii) is for about 24 hours to about 48 hours. In some aspects, the culturing of (iii) is for about 7 days. In some aspects, the passaging of (ii) is performed in a 2D matrix. In some aspects, the 2D matrix is Matrigel®. In some aspects, the passaging of (v) is performed in a 3D matrix. In some aspects, the 3D matrix is Matrigel®.

In some aspects, the differentiation methods provided herein comprise culturing cells in a base culture medium comprising a GSK3 inhibitor (e.g., CHIR99021), KGF, FGF10, dexamethasone, cAMP, and an inhibitor of cyclic nucleotide phosphodiesterases (e.g., IBMX), wherein the base culture medium does not comprise or is essentially free of a ROCK inhibitor. In some aspects, the culturing is for about 7 days.

In some aspects, the differentiation methods provided herein comprise:
(i) culturing LPC in a base culture medium comprising a GSK3 inhibitor (e.g., CHIR99021), KGF, FGF10, and a gamma secretase inhibitor (e.g., DAPT);
(ii) separating the cells of (i) having high expression of EpCAM and/or CPM;
(iii) passaging the cells of (ii) having high expression of EpCAM and/or CPM in a base culture medium comprising a GSK3 inhibitor (e.g., CHIR99021), KGF, FGF10, dexamethasone, cAMP, an inhibitor of cyclic nucleotide phosphodiesterases (e.g., IBMX), and a ROCK inhibitor (e.g., Y-27632);
(iv) culturing the cells of (iii) in a base culture medium comprising a GSK3 inhibitor (e.g., CHIR99021), KGF, FGF10, dexamethasone, cAMP, and an inhibitor of cyclic nucleotide phosphodiesterases (e.g., IBMX), wherein the base culture medium does not comprise or is essentially free of a ROCK inhibitor; and
(v) separating the cells of (iv) having high expression of SFTPC to form AT2 cells.

In some aspects, the passaging of (iii) is for about 24 hours to about 48 hours. In some aspects, the culturing of (iv) is for about 7 days. In some aspects, the passaging of (iii) is performed in a 3D matrix. In some aspects, the 3D matrix is Matrigel®.

In some aspects, the differentiation methods provided herein comprising passaging cells in a base culture medium comprising KGF, FGF10, dexamethasone, cAMP, an inhibitor of cyclic nucleotide phosphodiesterases (e.g., IBMX), and a ROCK inhibitor (e.g., Y-27632). In some aspects, such passaging is followed by culturing cells in a base culture medium comprising KGF, FGF10, dexamethasone, cAMP, an inhibitor of cyclic nucleotide phosphodiesterases (e.g., IBMX), and a GSK3 inhibitor (e.g., CHIR99021), wherein the base culture medium does not comprise or is essentially free of a ROCK inhibitor.

In some aspects, the differentiation methods provided herein comprise:
(i) culturing LPC in a base culture medium comprising a GSK3 inhibitor (e.g., CHIR99021), KGF, FGF10, and a gamma secretase inhibitor (e.g., DAPT);
(ii) passaging the cells of (i) in a base culture medium comprising a GSK3 inhibitor, KGF, FGF10, dexamethasone, cAMP, an inhibitor of cyclic nucleotide phosphodiesterases (e.g., IBMX), and a ROCK inhibitor (e.g., Y-27632);
(iii) separating the cells of (ii) having high expression of EpCAM and/or CPM;
(iv) passaging the cells of (iii) having high expression of EpCAM and/or CPM in a base culture medium comprising KGF, FGF10, dexamethasone, cAMP, an inhibitor of cyclic nucleotide phosphodiesterases (e.g., IBMX), and a ROCK inhibitor (e.g., Y-27632);
(v) culturing the cells of (iv) in a base culture medium comprising KGF, FGF10, dexamethasone, cAMP, an inhibitor of cyclic nucleotide phosphodiesterases (e.g., IBMX), and a GSK3 inhibitor (e.g., CHIR99021), wherein the base culture medium does not comprise a ROCK inhibitor; and
(vi) separating the cells of (v) having high expression of SFTPC to form AT2 cells.

In some aspects, the passaging of (iv) is from about 2 days to about 4 days. In some aspects, the culturing of (v) is from about 2 days to about 4 days. In some aspects, the passaging of (iv) is for about 2 days and the culturing of (v) is for about 2 days.

In some aspects, the passaging of (iv) and/or the culturing of (v) are repeated before the separating of (vi). In some aspects, the repeated passaging of (iv) is for about 4 days and/or the repeated culturing of (v) is for about 4 days. In some aspects, the passaging of (ii) is performed in a 2D matrix. In some aspects, the 2D matrix is Matrigel®. In some aspects, the passaging of (iv) is performed in a 3D matrix. In some aspects, the 3D matrix is Matrigel®.

### III. Other Aspects

The present disclosure is also related to AT2 cells made by any of the differentiation methods disclosed herein.

The present disclosure is also related to an organoid comprising the AT2 cells disclosed herein. As used herein, the term “organoid” refers to a differentiated or partially differentiated, 3D cellular organism derived from PSC (e.g., iPSC) or LPC which is self-organized by densely accumulating cells in a controlled space. Such organisms can be crafted to replicate much of the complexity of an organ, or to express selected aspects of it, for example, only producing certain types of cells.

Methods for maintaining differentiated AT2 cells and organoids are well known and include culturing the cells or organoids in a cell culture medium described herein and/or cryopreservation. Methods for making an organoid typically include culturing cells in a 3D matrix. Examples of a 3D matrix include, but are not limited to, polymers (natural or synthetic), ceramics, composites, or a combination thereof. A 3D matrix can be in the form of a hydrogel, a porous 3D scaffold, a rapid-prototyping scaffold, a foam, a sponge, a mesh, microparticles, fiber-like networks, mixtures of naturally occurring cell line-produced matrices such as Matrigel™, and combinations thereof, for example, microparticle-loaded hydrogels.

The present disclosure is also related to certain methods of using such AT2 cells and organoids. In some aspects, the present disclosure provides a method of treating a lung disease comprising administration of AT2 cells or an organoid made by any of the differentiation methods disclosed herein.

The present disclosure is also related to certain differentiation media for differentiating AT2 cells from PSC or LPC.

In some aspects, the differentiation medium comprises a base culture medium, a GSK3 inhibitor (e.g., CHIR99021), KGF, FGF10, and a gamma secretase inhibitor (e.g., DAPT). In some aspects, the differentiation medium comprises a base culture medium, from about 0.5 μM to about 6 μM of the GSK3 inhibitor (e.g., CHIR99021), from about 2 ng/mL to about 20 ng/mL KGF, from about 2 ng/mL to about 20 ng/mL FGF10, and from about 5 μM to about 40 μM of the gamma secretase inhibitor (e.g., DAPT). In some aspects, the differentiation medium comprises a base culture medium, about 3 μM of the GSK3 inhibitor (e.g., CHIR99021), about 10 ng/mL KGF, about 10 ng/mL FGF10, and about 20 μM of the gamma secretase inhibitor (e.g., DAPT).

In some aspects, the differentiation medium comprises a base culture medium, a GSK3 inhibitor (e.g., CHIR99021), KGF, FGF10, dexamethasone, cAMP, an inhibitor of cyclic nucleotide phosphodiesterases (e.g., IBMX), and a ROCK inhibitor (e.g., Y-27632). In some aspects, the differentiation medium comprises a base culture medium, from about 0.5 μM to about 6 μM of the GSK3 inhibitor (e.g., CHIR99021), from about 2 ng/mL to about 20 ng/mL KGF, from about 2 ng/mL to about 20 ng/mL FGF10, from about 20 nM to about 80 nM dexamethasone, from about 25 μM to about 200 μM of cAMP, from about 25 μM to about 200 μM of the inhibitor of cyclic nucleotide phosphodiesterases (e.g., IBMX), and from about 1 μM to about 20 μM of the ROCK inhibitor (e.g., Y-27632). In some aspects, the differentiation medium comprises a base culture medium, about 3 μM of the GSK3 inhibitor (e.g., CHIR99021), about 10 ng/mL KGF, about 10 ng/mL FGF10, about 50 nM dexamethasone, about 100 μM cAMP, about 100 μM of the inhibitor of cyclic nucleotide phosphodiesterases (e.g., IBMX), and about 10 μM of the ROCK inhibitor (e.g., Y-27632).

In some aspects, the differentiation medium comprises a base culture medium, a GSK3 inhibitor (e.g., CHIR99021), KGF, FGF10, dexamethasone, cAMP, and an inhibitor of cyclic nucleotide phosphodiesterases (e.g., IBMX). Optionally, the differentiation medium does not comprise or is essentially free of a ROCK inhibitor (e.g., Y-27632). In some aspects, the differentiation medium comprises a base culture medium, from about 0.5 μM to about 6 μM of the GSK3 inhibitor (e.g., CHIR99021), from about 2 ng/mL to about 20 ng/mL KGF, from about 2 ng/mL to about 20 ng/mL FGF10, from about 20 nM to about 80 nM dexamethasone, from about 25 μM to about 200 μM cAMP, and from about 25 μM to about 200 μM of the inhibitor of cyclic nucleotide phosphodiesterases (e.g., IBMX). In some aspects, the differentiation medium comprises a base culture medium, about 3 μM of the GSK3 inhibitor (e.g., CHIR99021), about 10 ng/mL KGF, about 10 ng/mL FGF10, about 50 nM dexamethasone, about 100 μM cAMP, and about 100 μM of the inhibitor of cyclic nucleotide phosphodiesterases (e.g., IBMX).

### EXAMPLES

Reference is now made to the following example, which together with the above descriptions illustrate some embodiments of the invention in a non-limiting fashion.

### Example 1

### Differentiation of LPC to AT2 Cells

An experiment was performed to differentiate alveolar type 2 (AT2) cells from lung progenitor cells (LPC) using the following protocol and reagents.

### 1. Distalization of LPC

The following reagents were used:
Iscove's Modified Dulbecco's Medium (IMDM) (Thermo Fisher Scientific; cat #12440053)
Hams F-12 Medium supplemented with L-Glutamine (Corning®; cat #10-080-CV)
GlutaMAX™ Supplement (Thermo Fisher Scientific; cat #35050061)
B-27 supplement (50×) (Thermo Fisher Scientific; cat #17504044)
Bovine serum albumin (BSA); 7.5% in Dulbecco's phosphate buffered saline (DPBS) (Sigma Aldrich; cat #A8412-100ML)
N-2 supplement (Thermo Fisher Scientific; cat #17502048)
L-ascorbic acid (Sigma-Aldrich; cat #A4544-25G)
1-thioglycerol (MTG) (Sigma-Aldrich; cat #M1753-100ML)
Primocin® (InvivoGen; cat #ant-pm-1)
IBMX (3-isobutyl-1-methylxanthine, Sigma-Aldrich; cat #15879)
8-bromoadenosine 3′,5′-cyclic monophosphate sodium salt (cAMP, Sigma-Aldrich; cat #B7880-100MG)
Dexamethasone powder (Sigma-Aldrich; cat #D4902-25 MG)
CHIR99021 (Biovision cat #1991-1)
Recombinant human keratinocyte growth factor (KGF; FGF-7) (Peprotech; cat #AF-100-19-50UG)
Recombinant human fibroblast growth factor-10 (FGF-10) (Peprotech; cat #AF-100-26-25UG)
DAPT (gamma secretase inhibitor IX) (Millipore Sigma #565770-10MG)
Dulbecco's modified eagle medium (DMEM)/F12 media (Thermo Fisher Scientific; cat #11320033)
Y-27632 (Rho-associated, coiled-coil containing protein kinase (ROCK) inhibitor) (STEMCELL™ Technologies; cat #72304)

Reagents were prepared as follows:
Ascorbic acid: A solution of 50 mg/mL ascorbic acid was made by dissolving 500 mg of ascorbic acid in tissue culture grade water. The solution was sterile-filtered and stored in 500 μL aliquots at −20° C. for up to 6 months.
MTG: A solution of 13 μL/mL MTG was made by adding 26 μL of MTG to 2 mL IMDM just before making complete serum-free differentiation media, discussed below.
Recombinant human KGF: KGF was reconstituted to a concentration of 0.1 mg/mL with sterile phosphate buffered saline (PBS) and diluted with 0.1% BSA/PBS to a concentration of 20 μg/mL. The solution was stored at −80° C. for up to 3 months.
Recombinant human FGF-10: FGF-10 was reconstituted to a concentration of 0.1 mg/mL with 5 mM sodium phosphate and diluted with 0.1% BSA/PBS to a concentration of 20 μg/mL. The solution was stored at −80° C. for up to 3 months.
DAPT: DAPT was reconstituted to a concentration of 20 mM in sterile dimethyl sulfoxide (DMSO). The solution was aliquoted and stored at −20° C. for up to 3 months.
Y-27632: Y-27632 was reconstituted to a concentration of 10 mM in sterile PBS.
Complete serum-free differentiation media (cSFDM): 375 mL of IMDM, 125 mL of Ham's F-12 media, 5 mL of GlutaMAX™ supplement, 5 mL of B-27 supplement, 3.3 mL of 7.5% BSA, 2.5 mL of N-2 supplement, 500 uL of 50 mg/mL ascorbic acid, 1.5 mL of 13 μL/mL MTG, and 500 μL of Primocin® were mixed and sterile-filtered. The media was stored in the dark at 4° C. for up to 1 month.
Distalization media (CFKD media): cSFDM was supplemented to a final concentration of 3 μM CHIR99021 (C), 10 ng/mL FGF-10 (F), 10 ng/mL KGF (K), and 20 μM DAPT (D).
BU-NGST, an induced pluripotent stem cell (iPSC)-derived reporter cell line developed by the Kotton Laboratory at Boston University was used. These cells have a green fluorescent protein (GFP) reporter and a TdTomato (TDT) reporter targeted to the Nkx2.1 locus and the surfactant protein C (SFTPC) locus, respectively. Carboxypeptidase M (CPM) can be used as an alternative for selection of Nkx2.1+ cells.

Distalization was performed by aspirating media from cells seeded in 6-well plates and adding 2 mL of CFKD media to each well. Plates were incubated at 37° C. for 24 hours. Media was changed in the same manner every 24 hours for 7 days.

### 2. Harvest Cells for 2D Passage and Quality Control

Next, cells were harvested for two-dimensional (2D) passage and quality control evaluation.

The following reagents were used:
Accutase™ (Biolegend; cat #423201)
Matrigel® Growth Factor Reduced (GFR) Basement Membrane Matrix, LDEV-free (Corning®; cat #354230)
Gentle cell dissociation reagent (GCDR) (STEMCELL™ Technologies, cat #07174)
IBMX (3-isobutyl-1-methylxanthine, Sigma-Aldrich; cat #15879)

Reagents were prepared as follows:
IBMX: IBMX was reconstituted to a concentration of 0.1 M in 200 proof ethanol. The solution was aliquoted and stored at −20° C. for up to one year.
10× cAMP/IBMX: A 10× cAMP/IBMX solution was made by mixing 50 mL cSFDM, 21.5 mg cAMP, and 500 μL of 0.1 M IBMX. The solution was sterile-filtered with a 0.22 μM filter and stored at 4° C. for up to 1 month in the dark.
Dexamethasone: Dexamethasone was reconstituted to a concentration of 1 mM in 63.7 mL of molecular biology grade ethanol. The solution was stored at −20° C. for up to 2 years. A solution of 100 μM dexamethasone was then made by mixing 500 μL of 1 mM dexamethasone and 4.5 mL molecular biology grade ethanol. The solution was aliquoted in 100 μL volumes and stored at −20° C. for up to one year.
Y-27632: Y-27632 was reconstituted to a concentration of 10 mM in PBS. The solution was aliquoted and stored at −20° C. for up to 6 months.
CK+DCI+F media: cSFDM media was supplemented with 3 μM CHIR99021 (C), 10 ng/mL KGF (K), 10 ng/mL FGF-10 (F), 50 nM dexamethasone (D), and 1× cAMP/IBMX (CI). On the day of cell passaging or seeding, the media was supplemented with 10 μM Y-27632.

### Matrigel® Plate Coating Preparation

Matrigel® was thawed overnight on ice and aliquoted into 1.5 mL microfuge tubes according to the dilution factor provided by the manufacturer. Aliquots were stored at −20° C. The day of cell seeding, Matrigel® was diluted with cold DMEM/F-12 media according to the dilution factor provided by the manufacturer so that the volume is sufficient to coat the wells/plates used for cell seeding. Next, the wells of 6-well plates were coated with 1.5 mL Matrigel®/DMEM F-12 solution per well and incubated for at least 1 hour at 37° C. The wells were rinsed with 1 mL of DMEM/F-12 media before cells were added.

### Harvest Cells for Passage and Flow Cytometry QC

Media was removed from 6-well plates and wells were washed with 2 mL of PBS. The PBS was removed and 1.5 mL of Accutase™ was added to each well. Plates were incubated for about 15-20 minutes at 37° C. or until cells easily detached. Harvested cells were diluted with an equal volume of DMEM/F12 media supplemented with 0.25% BSA. Cells were pipetted up and down with a p1000 pipettor to dissociate cell clumps into single cells.

Cells were then filtered with a 30 μM filter to remove debris and spun at 200 g for 5 minutes. The media was aspirated and approximately 2-3 mL of CK+DCI+F media with 10 μM Y-27632 was added. Cells were pipetted up and down with a p1000 pipettor to dissociate cell clumps into single cells, and counted with a Moxi™ cell counter (ORFLOW® Technology). More CK+DCI+F media with 10 μM Y-27632 was added so that the cell concentrations were between approximately 1-2 million cells/mL.

Approximately 250,000 to 300,000 cells were set aside for flow cytometry quality control (QC) assays. The remaining cells were spun down and resuspended to a concentration of approximately 1 million cells per mL in DMEM/F12 media supplemented with 10% fetal bovine serum (FBS). Cells were seeded into Matrigel®-coated 6-well plates at approximately 208,000 cells/cm².

Following incubation, media was aspirated and 2 mL of CK+DCI+F media was added to each well. Plates were incubated at 37° C. for 24 hours. For the first 24-48 hours of cell seeding, Y-27632 was added to the media (10 μM final concentration). Media was changed in the same manner every 24 hours for 7-10 days.

3. Sort Based on EpCAM+/GFP with FACS and Passage to 3D

Next, cells were sorted based on the expression of EpCAM/GFP or EpCAM/CPM with fluorescence-activated cell sorting (FACS). A media was made for sorting by supplementing cSFDM with 10 ng/mL KGF (K), 10 ng/mL FGF-10 (F), 50 nM dexamethasone (D), 1× cAMP/IBMX (CI), and 10 μM Y-27632 (Y) (FK+DCI+Y media).

Media was removed from the seeded plates above and 2 mL of PBS was added to each well. PBS was removed and 1.5 mL of Accutase™ was added to each well. Plates were incubated for approximately 15-20 minutes at 37° C. or until cells were easily detached. The harvested cells were diluted in an equal volume of DMEM/F12 media supplemented with 0.25% BSA. The cells were pipetted up and down with a p1000 pipettor to dissociate cell clumps into single cells. Cells were filtered through a 30 μM filter to remove debris and cells were spun at 200 g for 5 minutes. Media was removed and DMEM/F12 media with BSA or LPC sort buffer was added. Cells were counted with a Moxi™ cell counter (ORFLOW® Technology). Approximately 250,000-300,000 cells were set aside for flow cytometry QC assays. The remaining cells were spun down and resuspended at approximately 1 million cells/mL in DMEM/F12 media supplemented with 10% FB S.

Cells were then sorted based on GFP expression using a cell sorter from Sony Biotechnology (MA900). BU-NGST iPSC (CL00366) and BU-NGST LPC were used as control cells. The cells were spun down and resuspended in 1:750 Ghost Dye™ Red780/PBS and incubated for 10 minutes in the dark. An equal volume of DMEM/F12 supplemented with 10% FBS was then added to neutralize the Ghost Dye™. Cell were spun down for 5 minutes at 200 g at 4° C. and resuspended in LPC FACS Sort Buffer. LPC were then sorted based on single cell gates (FSC-H vs. FSC-w and SSC-H vs. SSC-W); live cells (Ghost Dye™ Red780 low) in APC-Cy7 channel; GFP+ and purity or semi-purity mask for the desired number of cells.

### 4. Distalized LPC Passage to 3D Matrigel® Culture

After collecting the sorted LPC, the cells were centrifuged 200 g for 5 minutes at 4° C. The media was aspirated from the cell pellet. Undiluted Matrigel® Growth Factor Reduced (GFR) basement membrane matrix or Matrigel® for Organoid Culture (Corning®) was added to obtain a density of approximately 350-400 cells/μL and the cells were resuspended. Approximately 45-50 μL of the suspension was added to each well of a 12-well plate. The suspension was allowed to solidify for at least 20 minutes at 37° C. After the suspension was fully polymerized, 1 mL of pre-warmed FK+DCI+Y media was added to each well.

Cells began to form epithelial spheres (spheroids) within several days to one week of culture. Spheroids were grown for 10-12 days until they reached a size of approximately 100-150 μm.

### 5. 3D Culture, CHIR99021 (CHIR) Cycling Media Change Protocol

Cells were kept in FK-DCI+Y media for 48 hours. Then, CHIR99021 was added back to the media and Y-27632 was removed (FK-DCI+C media). After 48 hours in this media, a cycle of incubation in media without CHIR99021 for 4 days was performed, followed by incubation in media with CHIR99021 for 4 days.

Specifically, media was carefully aspirated from each well of the 12-well plates, and approximately 1-1.2 mL of media (with or without CHIR99021) was added to each well. Plates were incubated at 37° C. for 48 hours. This media change was repeated every other day, using the “CHIR cycling” method described above, starting with 2 days off (CHIR99021 absent) and 2 days on (CHIR99021 present) and followed by 3-4 days off and 3-4 days on until passage.

6. Harvesting AT2 from 3D Matrigel® Culture for Passage and QC (Day −40)

The following reagents were used:
Dispase II (Thermo Fisher Scientific; cat #17105041). Dispase II was made 2 mg/mL DMEM medium. The media as sterile filtered and stored at 4° C. for up to 2 weeks or aliquoted and froze at −20° C. for up to 2-3 months.
TrypLE™ Express (Thermo Fisher Scientific; cat #12604013)
Matrigel® Growth Factor Reduced (GFR) Basement Membrane Matrix, LDEV-free (Corning®; cat #354230)
Matrigel® for Organoid Culture (Corning® #356255)

### 7. 3D Matrigel® Culture Harvest Protocol

Media was aspirated from plates, and 1 mL of 2 mg/mL Dispase II was added. Plates were incubated at 37° C. for about 1 hour until the Matrigel® was fully dissolved. The Matrigel® pellet was gently dislodged 10 minutes into incubation, and gently pipetted 3-5 times every 10 minutes to facilitate dissociation. Cells were transferred to a new 15 mL conical tube and centrifuged at 200 g for 2-3 minutes at 8° C. The supernatant was aspirated and an appropriate amount of TrypLE™ Express was added to resuspend all the cells/spheroids. Cells were incubated for about 15 minutes at 37° C. with occasional pipetting every 5 minutes. A 1:1 volume of DMEM/F12+0.25% BSA was then added to dilute and inactivate the Tryp™LE. Cells were filtered a 30 μM filter.

Cells were then counted with a Moxi™ cell counter (ORFLOW® Technology). Approximately 250,000-300,000 cells were removed for FACS QC. Cells were centrifuged and resuspended in 1M/m: in DMEM/F12+10% FBS. Cells were passaged on for downstream AT2 analysis with flow cytometry. For the remaining cells, the following sorting protocol was used. On the first passage that SFPTC+ cells make up greater than 15% of the GFP+ cells, sort on TDT into 3D culture.

### 8. Sorting Protocol for AT2 (SFPTC+) Organoids

Organoids based on TDT+ were sorted with a cell sorter from Sony Biotechnology (MA900). The sorter was calibrated and initialized according to manufacturer's standard operating protocol. The following control cells were thawed and prepared: BU-NGST iPSC (CL00366) or BU-NGST LPC for GFP control.

Cells were centrifuged, resuspended in 1:750 Ghost Dye™ Red 780/PBS, and incubated for 10 minutes in the dark. An equal volume of DMEM/F12+10% FBS was then added to neutralize Ghost Dye™. Cells were centrifuged and resuspended LPC FACS Sort Buffer. LPC were sorted into LPC FACS Sort Buffer based on: Single cells gates (FSC-H vs. FSC-w and SSC-H vs. SSC-W); live cells (Ghost Dye Red780 low) in APC-Cy7 channel; GFP+/TDT+; and purity or semi-purity mask for the desired number of cells.

Cells were then plated in 50 μL of Matrigel® GFR or Matrigel® for Organoid Culture for further 3D culture at a cell density of 350-400 cells/μL.

### 9. Culture and Passage of AT2 Organoids

After TDT+ sort and a second passage, the TDT+ organoid population was greater than 50%. Cells were passaged in 3D culture with continued cycling of CHIR99021 (CHIR cycling), as described above.

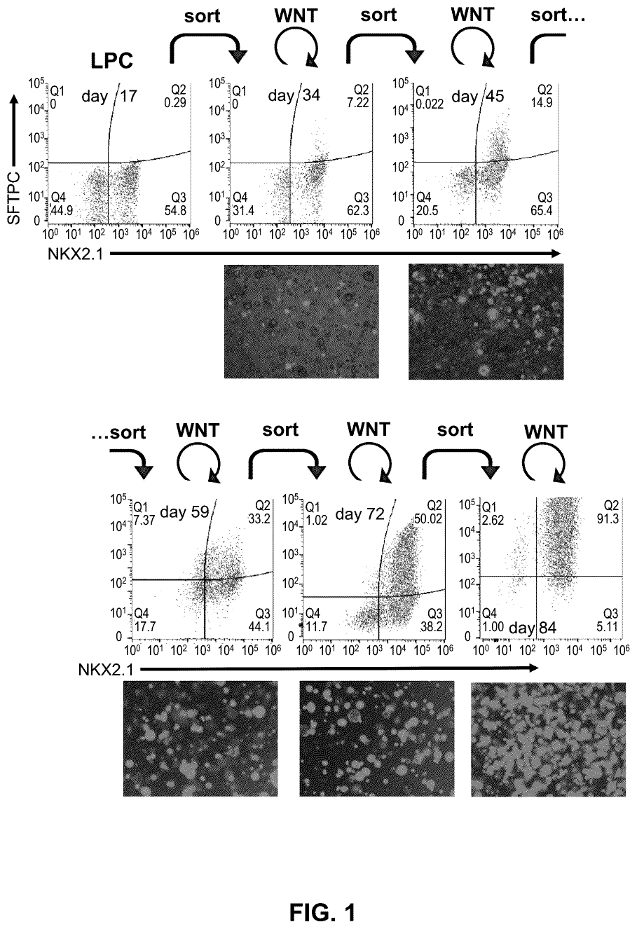

Expression of the SFTPC reporter was evaluated during differentiation. Flow cytometry results from day 17, day 34, day 45, day 59, day 72 and day 84 are shown in FIG. 1. Differentiated cells exhibited robust SFTPC reporter expression, indicating promoter activation. Cells required continued cycling of CHIR99021 to reinforce the AT2 program and gain stability with Nkx2.1 and SFTPC expression.

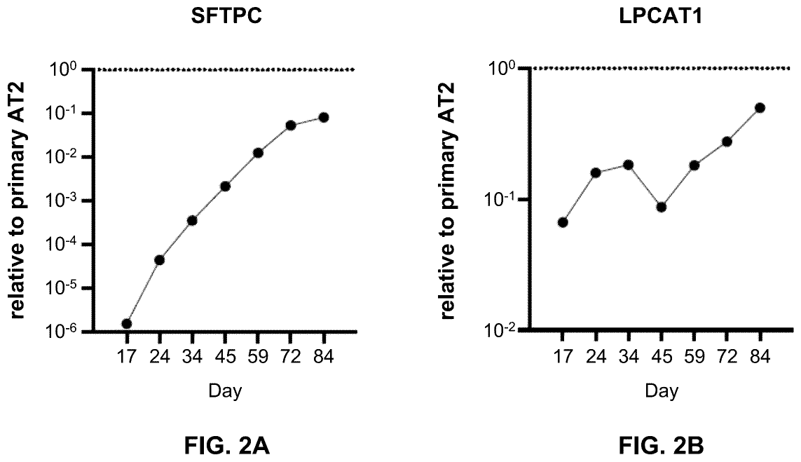

Expression of AT2-associated genes during the differentiation protocol was also evaluated relative to a primary AT2 cell control. The results are shown in FIG. 2A-2B for the genes SFTPC (FIG. 2A) and LPCAT1 (FIG. 2B). Differentiated cells exhibited robust induction of AT2-associated genes over time.

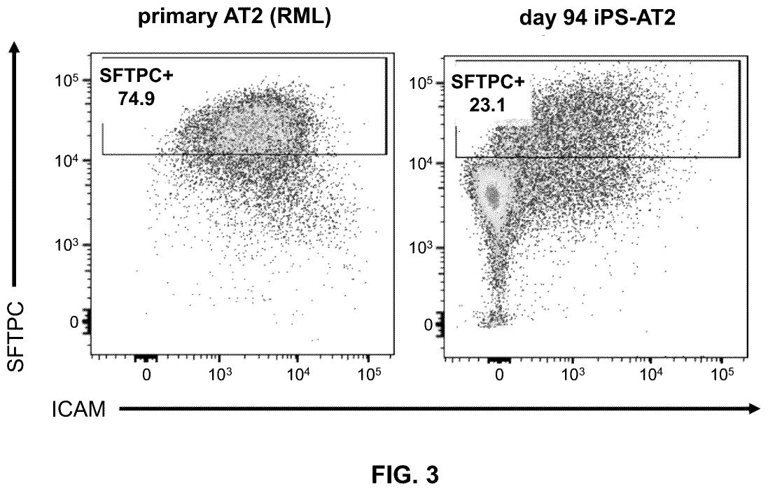

SFTPC protein was also detected in differentiated cells by flow cytometry. Flow cytometry results from day 94 of differentiated cells (iPS-AT2) are shown in FIG. 3, along with results from control primary AT2 cells.

All publications, patents and patent applications mentioned in this application are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method for differentiating lung progenitor cells (LPC) into alveolar type 2 (AT2) cells, comprising:
culturing LPC in a base culture medium comprising a glycogen synthase kinase 3 (GSK3) inhibitor, keratinocyte growth factor (KGF), fibroblast growth factor 10 (FGF10), and a gamma secretase inhibitor;
(ii) passaging the cells of (i) in a base culture medium comprising a GSK3 inhibitor, KGF, FGF10, dexamethasone, cyclic adenosine monophosphate (cAMP), an inhibitor of cyclic nucleotide phosphodiesterases, and a Rho-associated, coiled-coil containing protein kinase (ROCK) inhibitor for about 24 hours to about 48 hours;
(iii) culturing the cells of (ii) in a base culture medium comprising a GSK3 inhibitor, KGF, FGF10, dexamethasone, cyclic adenosine monophosphate (cAMP), and an inhibitor of cyclic nucleotide phosphodiesterases for about 7 days;
(iv) separating the cells of (iii) having expression of epithelial cellular adhesion molecule (EpCAM) and/or carboxypeptidase M (CPM);
(v) passaging the cells of (iv) having expression of EpCAM and/or CPM in a base culture medium comprising a GSK3 inhibitor, KGF, FGF10, dexamethasone, cAMP, and an inhibitor of cyclic nucleotide phosphodiesterases, and a ROCK inhibitor; and
(vi) separating the cells of (v) having expression of surfactant protein C (SFTPC) to form AT2 cells.

2. The method of claim 1, wherein the passaging of (ii) is performed in a two dimensional (2D) matrix.

3. (canceled)

4. The method of claim 1, wherein the passaging of (v) is performed in a three dimensional (3D) matrix.

5. (canceled)

6. A method for differentiating LPC into AT2 cells, comprising:
(i) culturing LPC in a base culture medium comprising a GSK3 inhibitor, KGF, FGF10, and a gamma secretase inhibitor;
(ii) separating the cells of (i) having expression of EpCAM and/or CPM;
(iii) passaging the cells of (ii) having expression of EpCAM and/or CPM in a base culture medium comprising a GSK3 inhibitor, KGF, FGF10, dexamethasone, cAMP, an inhibitor of cyclic nucleotide phosphodiesterases, and a ROCK inhibitor for about 24 hours to about 48 hours;
(iv) culturing the cells of (iii) in a base culture medium comprising a GSK3 inhibitor, KGF, FGF10, dexamethasone, cAMP, and an inhibitor of cyclic nucleotide phosphodiesterases for about 7 days, wherein the base culture medium does not comprise or is essentially free of a ROCK inhibitor; and
(v) separating the cells of (iv) having expression of SFTPC to form AT2 cells.

7. The method of claim 6, wherein the passaging of (iii) is performed in a 3D matrix.

8. (canceled)

9. A method for differentiating LPC into AT2 cells, comprising:
(i) culturing LPC in a base culture medium comprising a GSK3 inhibitor, KGF, FGF10, and a gamma secretase inhibitor;
(ii) passaging the cells of (i) in a base culture medium comprising a GSK3 inhibitor, KGF, FGF10, dexamethasone, cAMP, an inhibitor of cyclic nucleotide phosphodiesterases, and a ROCK inhibitor;
(iii) separating the cells of (ii) having expression of EpCAM and/or CPM;
(iv) passaging the cells of (iii) having expression of EpCAM and/or CPM in a base culture medium comprising KGF, FGF10, dexamethasone, cAMP, an inhibitor of cyclic nucleotide phosphodiesterases, and a ROCK inhibitor;
(v) culturing the cells of (iv) in a base culture medium comprising KGF, FGF10, dexamethasone, cAMP, an inhibitor of cyclic nucleotide phosphodiesterases, and a GSK3 inhibitor, wherein the base culture medium does not comprise or is essentially free of a ROCK inhibitor; and
(vi) separating the cells of (v) having expression of SFTPC to form AT2 cells.

10. The method of claim 9, wherein the passaging of (iv) is from about 2 days to about 4 days and/or the culturing of (v) is from about 2 days to about 4 days.

11. (canceled)

12. The method of claim 9, wherein the passaging of (iv) and/or the culturing of (v) are repeated before the separating of (vi).

13. (canceled)

14. The method of claim 9, wherein the passaging of (ii) is performed in a 2D matrix.

15. (canceled)

16. The method of claim 9, wherein the passaging of (iv) is performed in a 3D matrix.

17. (canceled)

18. The method of claim 1, wherein the GSK3 inhibitor is CHIR99021.

19. The method of claim 18, wherein CHIR99021 is present in the culture medium at a concentration of about 3 μM.

20. The method of claim 1, wherein FGF10 is present in the culture medium at a concentration of about 10 ng/mL.

21. The method of claim 1, wherein KGF is present in the culture medium at a concentration of about 10 ng/mL.

22. The method of claim 1, wherein the gamma secretase inhibitor is N-[N-(3,5-difluorophenacetyl)-1-alanyl]-s-phenylglycinet-butyl ester (DAPT).

23. The method of claim 22, wherein DAPT is present in the culture medium at a concentration of about 20 μM.

24. The method of claim 1, wherein dexamethasone is present in the culture medium at a concentration of about 50 nM.

25. The method of claim 1, wherein cAMP is present in the culture medium at a concentration of about 100 μM.

26. The method of claim 1, wherein the inhibitor of cyclic nucleotide phosphodiesterases is 3-isobutyl-1-methylxanthine (IBMX).

27. The method of claim 26, wherein IBMX is present in the culture medium at a concentration of about 100 μM.

28. The method of claim 1, wherein the ROCK inhibitor is Y-27632.

29. The method of claim 28, wherein Y-27632 is present in the culture medium at a concentration of about 10 μM.

30. AT2 cells made by the method of claim 1.

31. An organoid comprising the AT2 cells of claim 30.

32-33. (canceled)

34. A differentiation medium comprising a base culture medium, KGF, FGF10, dexamethasone, cAMP, an inhibitor of cyclic nucleotide phosphodiesterases, and a ROCK inhibitor.

35. (canceled)
